(19) United States
(12) Patent Application Publication
(10) Pub. No.: US 2020/0121211 A1
(43) Pub. Date: Apr. 23, 2020

(54) EKG/ECG Lead wire detangler box

(71) Applicant: Helen Louise Tutino, Catonsivlle, MD (US)

(72) Inventor: Helen Louise Tutino, Catonsivlle, MD (US)

(21) Appl. No.: 16/350,265

(22) Filed: Oct. 22, 2018

(51) Int. Cl. A61B 5/0428; A61B 5/0404

(52) U.S. Cl. CPC A61B 5/04286; A61B 2562/222; A61B 5/0404

(57) ABSTRACT

A medical device that is essentially a box with separate chambers to be used in conjunction with a typical 5 lead or 12 lead EKG/ECG wire set to prevent the EKG/ECG wires from tangling by keeping the wires separated and when used as intended will aid in rapid easy proper placement of the lead wires on the human body to expedite diagnostic results.

### BACKGROUND OF THE INVENTION

I am a nurse who worked in the cardiovascular intensive care unit and all of my patients were having heart attacks. One element of the job that was very stressful for me was trying to untangle the EKG lead wires prior to placing the leads on the patient. I spent precious time untangling lead wires in front of a patients having a heart attacks and thought there must be a better way to get these on faster. I have done a Google search for EKG separators and I have seen spool mechanisms and small plastic pieces which separate the wires but these seemed very complicated or too simple.

### BRIEF SUMMARY OF THE INVENTION

This is a medical device that is essentially a mostly rectangular box that when closed creates 10 separate chambers to be used to separate the wires of any typical 5 lead or 12 lead EKG/ECG lead wire set to keep them separated and untangled and if stored in the proper way will allow for fast easy proper placement of the EKG/ECG lead wires on an individual. It also serves to keep these wires separated and reduce tangles while not in use. This device is easier to use than the existing devices. Existing devices require the turning a spool mechanism and winding of the woes or small plastic pieces that are used to separate wires but these small devices are often lost and rarely used. This device is larger and more practical than the existing ones that serve a similar purpose.

### BRIEF DESCRIPTION OF THE DRAWINGS

In the following detailed portion of the present description. The details are explained in the detailed description of the invention section with reference to the example shown in the drawings which include:

FIG. 1 depicts the top of the device laying on one of the larger flat sides. This side is intended for use with a 12 lead EKG/ECG lead wire device and depicts a torso with the suggested placement for the wires (device designed to use with EKG/ECG lead wires). The lower part of the image illustrates the 10 key hole cut out areas at the outer edge. The intended use for these areas is to allow select wires to be squeezed or pinched into the chambers leaving most of the wire exposed on the outside of the device leaving only a small section of the wire sitting in the chamber. One side shows the hinge, the other shows the snap clasp used for closing.

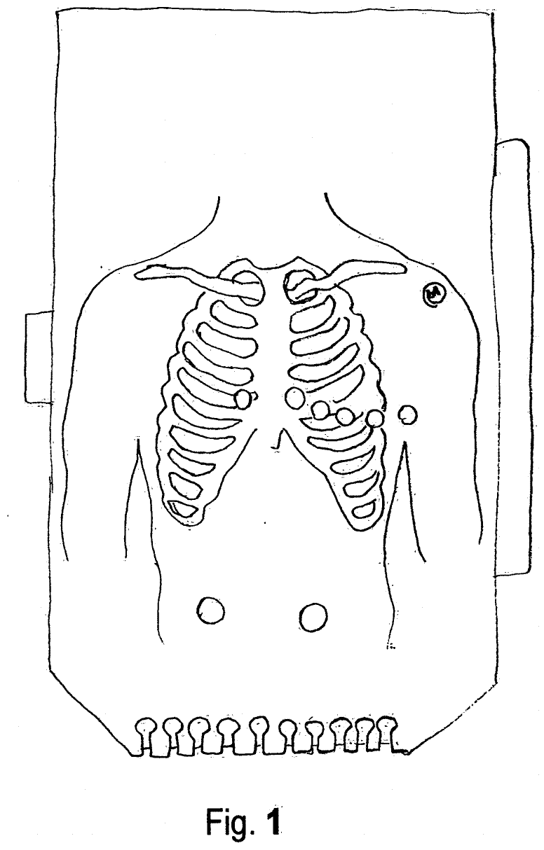

FIG. 2 illustrates the opposite side of FIG. 1 and depicts the top of the device laying on one of the larger flat sides. This side is intended for use with a 5 lead EKG/ECG lead wire device and depicts a torso with the suggested placement for these wires (device designed to use with EKG/ECG lead wires). The lower part of the image illustrates the 4 key hole cut out areas at the outer edge. The intended use for these areas is to allow select wires to be squeezed or pinched into the chambers leaving most of the wire exposed on the outside of the device leaving only a small section of the wire sitting in the chamber. One side shows the hinge, the other shows the snap clasp used for closing.

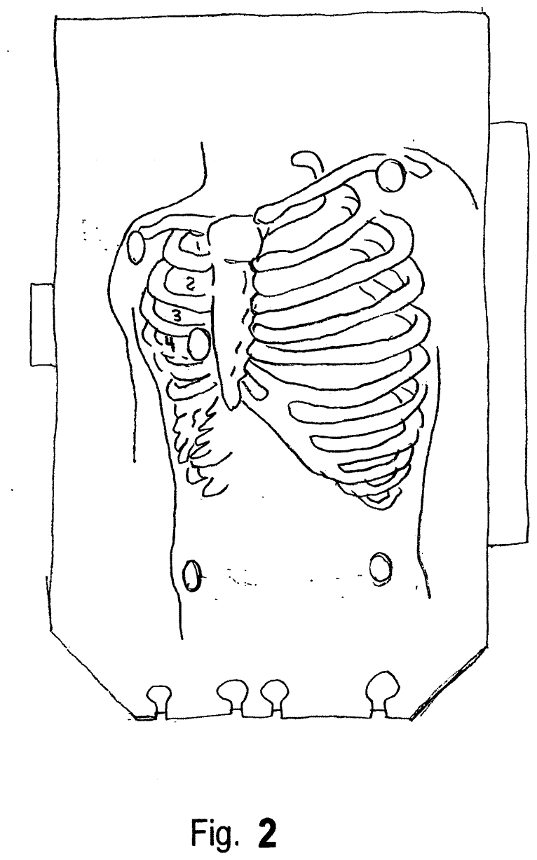

FIG. 3 illustrates the device as seen from the side while closed and reveals the hinged side and an angle in the one end of the device.

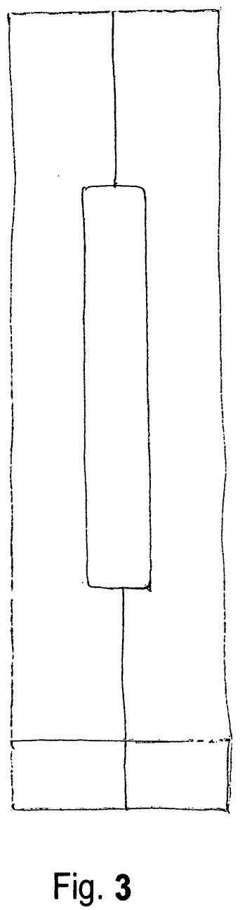

FIG. 4 illustrates the device as seen from the side and reveals the snap clasp closure device and an angle in the one end of the device.

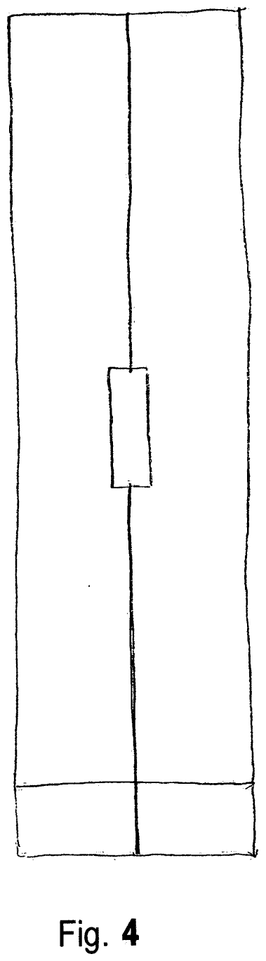

FIG. 5 illustrates the device opened all the way by the hinge with dividers creating 10 channels or compartments on each side. The top of the angled side reveals 14 small key hole cut outs. The key holes are different on each side as one is intended for a 12 lead wire placement and the other side when in use with the 5 leads. The sides show the closures.

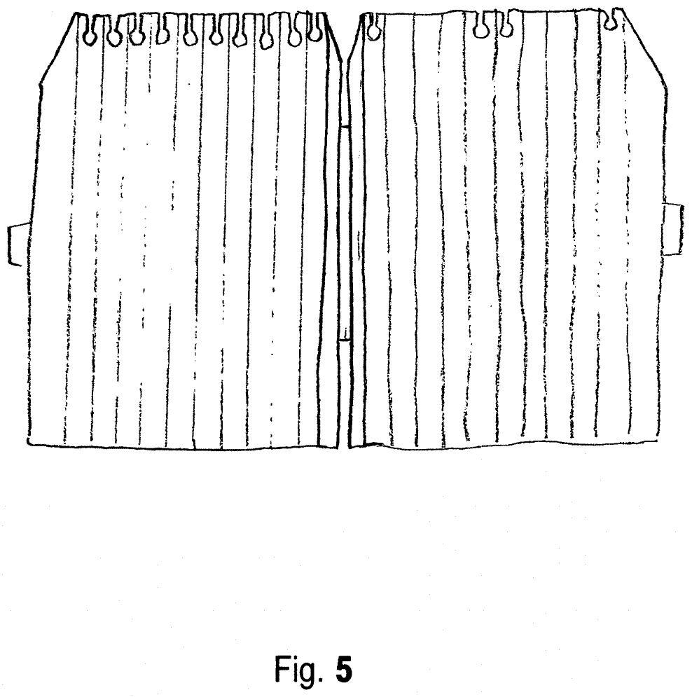

FIG. 6 illustrates the device closed observed if viewing at a 45 degree angle and the closest point was the bottom corner of the device. This image shows the 10 chambers created when the when device is snapped closed. The top end of the device illustrates the 10 key hole shapes for select wires to project through and the internal divides on the inside that create the 10 long chambers to hold wires when the device is closed that will extend out at the bottom.

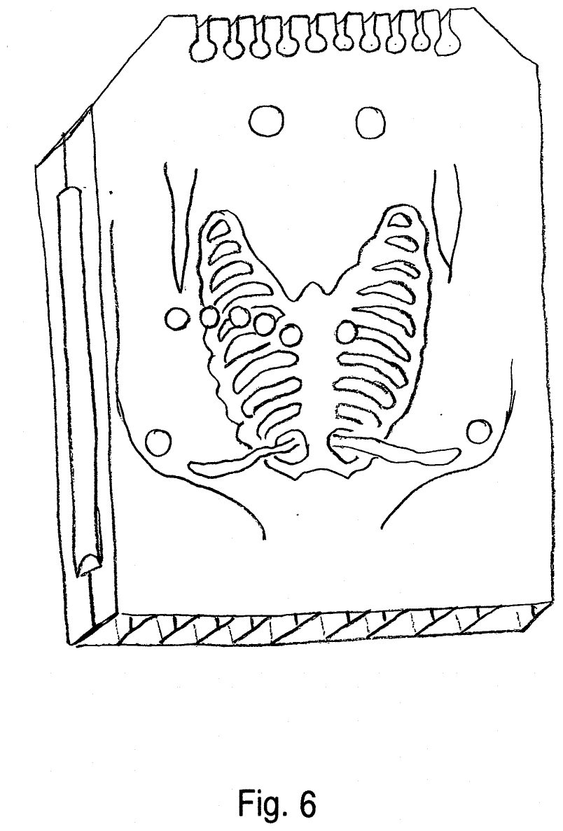

FIG. 7 Illustrates the device with the wire leads device it is intended to be used in conjunction with.

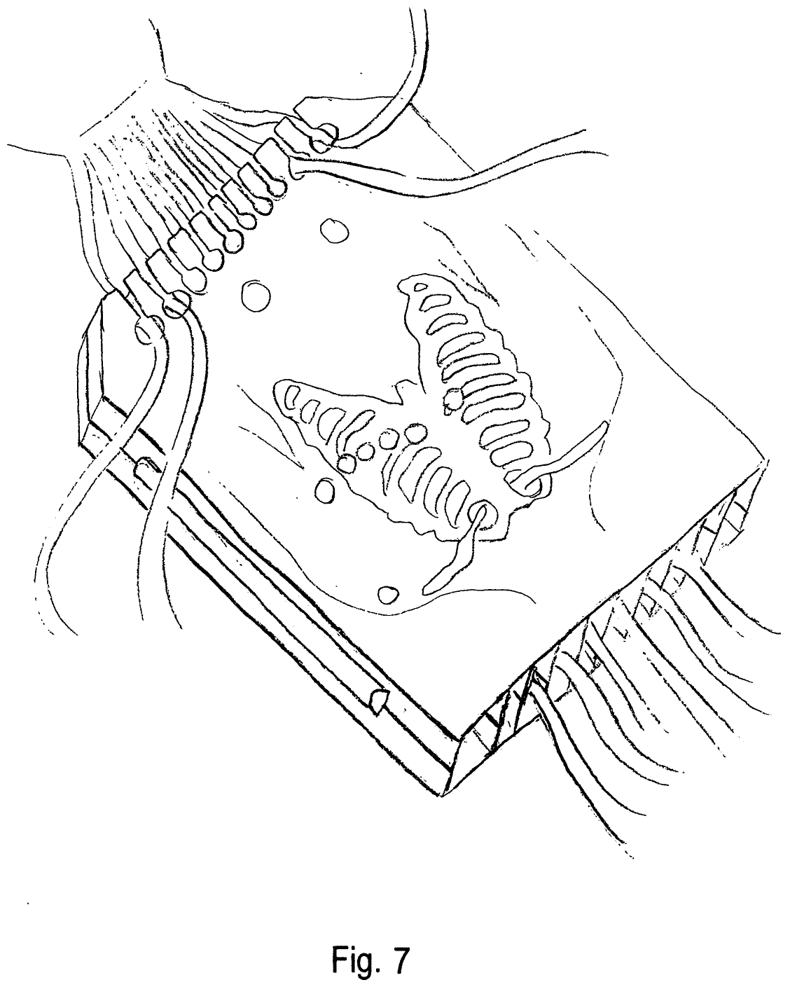

FIG. 8 illustrates the device open.

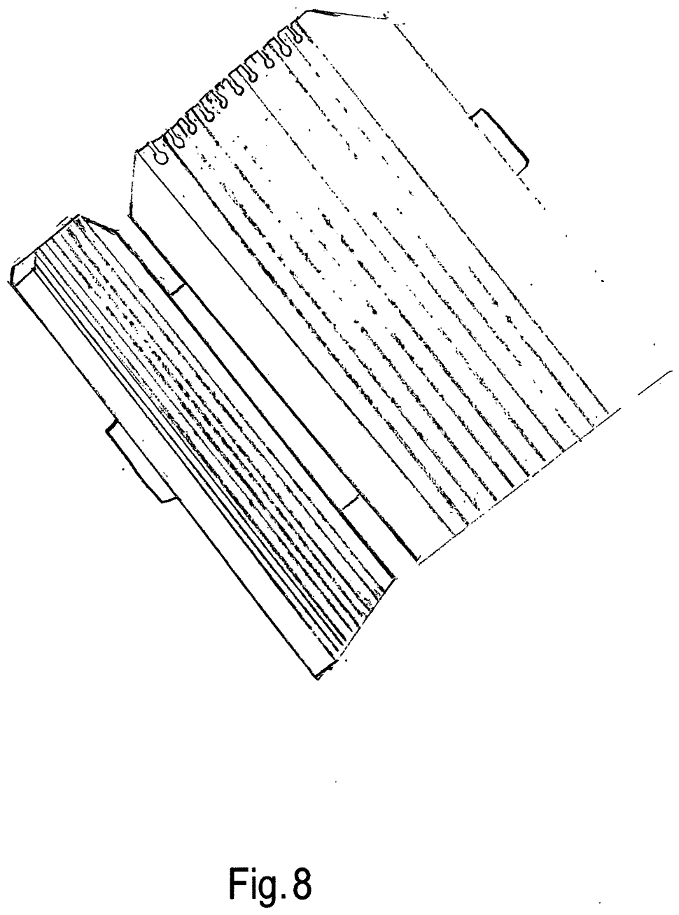

FIG. 9 illustrates one side of the device to show the inside dividers which create the chambers.

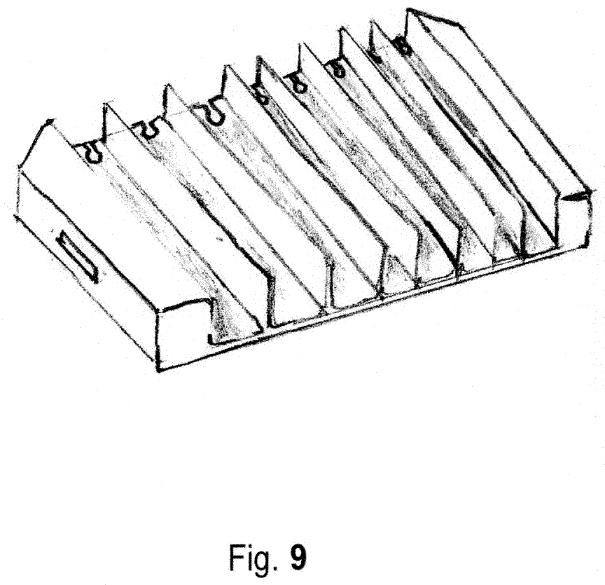

### DETAILED DESCRIPTION OF THE INVENTION

This is a medical device that is to be used with a typical 5 lead or 12 lead EKG/ECG lead wire set to keep the wires separated or prevent tangled wires, and when stored in the proper way will allow for fast easy proper placement of the leads on the patient. The device can be slid along the wires to also resolve tangles. It is made of anti-microbial plastic/rubber and is easily removed for cleaning. It can be used in conjunction with either a 5 lead or 12 EKG/ECG wire set including different brands of wire sets. To aid the user, it contains the image of a torso with bones and circles that depict the recommended placement of a 5 lead EKG/ECG on one side's face, and the recommended placement of a 12 lead EKG/ECG on the other side's face. When the device is placed on the patient's chest the image on the device (when placed to reflect the position of the body lying on the bed and the number of leads in use either 5 or 12) will allow the leads to be in close proximity to where they should be secured on the body. When the lead wires are pulled from the box (device), the box easily slides further down the wires and out of the way. The device should be stored snapped onto all the EKG lead wires and slid down as far as it can go or until it catches on the clip of the shortest wire (most EKG/ECG's have a clip to attach to an electrode), so that the wires that attach to the body are in the proper place and ready for use. If stored properly this device prevents tangles and will save time and aid with easy placement of EKG/ECG wires on a body.

This device is primarily a rectangular box shape with a trapezoid shape on one end. It has a hinge and a snap closure mechanism to open and close the device. There are internal dividers on each side of the box which create 10 separate hollow chambers within the device when the device is snapped closed. These chambers are where the wires of and EKG/ECG device are placed when in use. One end of the box is shaped differently than the other to allow visual identification of each side. Also, so that the device is more compatible with most EKG/ECG wire products it can be used with. The material is a semi-flexible plastic/rubber and is light weight. This device has stickers for labeling the inside and outside of the device that allow use with many EKG/ECG wire brands.

Atop the 10 hollow chambers, which run the entire length of the device, starting at the edge of the trapezoid side, are small open keyhole shaped areas on the face of both the top and bottom on each face (2 largest flat sides). These open keyhole areas begin at the edge and extend a short distance to create a small gap down the middle of the top of the hollow chambers that finish in an oval shaped hole. These open keyhole areas allow a wire to be placed into a chamber and emerge from the device after a short distance to leave the remainder of the chamber empty. These keyhole shaped areas allow for quick removal and placement of the longer EKG/ECG lead wires. These open keyhole areas allow the wires to be easily removed from the box but keep them in the proper place for use. Typically the longer lead EKG/ECG wires go on the legs and arms and these wires would not need to run through the whole length of the chamber to exit at the bottom of the device like the other wires. The mechanics of the open keyhole pinch areas are similar to a vacuum cleaners plug wire which can be pinched onto itself for storage. It is also similar to the mechanics of a bangle bracelet. A person would turn the wrist sideways to remove a wide bangle bracelet made of metal; only in this device the compartment is made of flexible plastic/rubber so the lead wires can be pinched or squeezed into each chamber and easily pulled out. Another example would be saloon bar doors that can be pushed through but swing closed.

The primary use of the device is to keep lead wires separated and untangled and it is easier to use than the existing devices which require the turning a spool mechanism and the winding of wires and this device is larger and more practical than the existing small plastic pieces that are used to separate wires currently, because too often these little plastic pieces are either lost or rarely used.

1. A device comprising a box that creates separated chambers or channels, wherein wires of an EKG/ECG lead wire set can be placed to keep the wires separated to reduce or prevent tangled wires and slid along wires to resolve tangles.

2. A device according to claim 1, wherein the body is asymmetrical in shape and the exterior faces of the top and bottom of the operational box displays artwork of a torso with bones and circles to denote proper placement of either a 5 lead or 12 lead set of wires for an EKG/ECG to assist the user with placement of the wires on a human body.

3. A device according to claim 1, wherein the essential box shape with hollow channels can be slid along lead wires of an EKG/ECG device to prevent tangled wires.

3. A device according to claim 1, wherein the keyhole cut out shapes on the device function to allow a wire to be placed into the chamber by being pinched or squeezed into that chamber for only a short distance and to exit the top of the device, rather than the end of the device.

4. A device according to claim 2, wherein one end of the device is a different (trapezoid) shape than the other end to aid in proper placement by allowing the user to more easily recognize one end of the device from the other.

4. A device according to claim 1, wherein the operational box is made of anti-microbial plastic/rubber to allow for flexibility especially in the keyhole areas of the device designed to allow fast easy placement and removal of wires.

5. A device according to claim 2, wherein the operational box displays artwork of a torso with bones and lead placement circles and is to include stickers with labels for use on the inside and outside of the device to be placed by the user to assist with placement of leads used with this device.

6. A device according to claim 1, wherein the operational box when snapped closed, with lead wires within the chambers, can be slid up and down the wires to prevent/reduce or untangle wires.

7. A device according to claim 1, wherein if stored snapped with lead wires placed in appropriate chambers and the device is slide down as far as possible and stopped by the first clip of the shortest lead wire (of the typical 5 lead or 12 lead EKG/ECG wire set the device is meant to work in conjunction with) will prevent tangles and expedite the placement of lead wires.

8. A device according to claim 2, when placed on a human body with the artwork mirroring the position of the body with the appropriately facing EKG/ECG lead number (5 or 12) image displayed on the face of the device aids the user in the placement of the lead wires.
